(19) United States
(12) Patent Application Publication
(10) Pub. No.: US 2003/0083209 A1
(43) Pub. Date: May 1, 2003

(54) Viscosity modification of petroleum distillates

(76) Inventor: Timothy I. Moodycliffe, Milwaukee, WI (US)

Correspondence Address: S.C. JOHNSON & SON, INC., 1525 HOWE STREET, RACINE, WI, 53403-2236, US

(21) Appl. No.: 10/007,496

(22) Filed: Oct. 22, 2001

(51) Int. Cl. A61K 7/047

(52) U.S. Cl. 510/118; 510/136; 510/185; 510/477

(57) ABSTRACT

A method for the thickening of mineral oil products, wherein the thickened mineral oil product exhibits shear-thinning so as to be applicable to a surface by non-pressurized spray mechanisms. The thickening agent added to the petroleum distillate comprises a thixotropic thickener chosen from the group consisting of mixtures comprising a triblock copolymer and a hydrocarbon oil, and N-Lauroyl glutamic acid di-n-butylamide.

### RELATED APPLICATION(S)

Not applicable.

### FEDERALLY SPONSORED RESEARCH OR DEVELOPMENT

Not applicable.

### BACKGROUND OF THE INVENTION

### FIELD OF THE INVENTION

The present invention relates to a method for the preparation of petroleum distillate products having a modified viscosity, but with shear properties appropriate for dispensing as a spray by means of spray bottles or pump apparatus.

### BACKGROUND INFORMATION

Petroleum distillate products are known for use in any number of products, including such common household products as baby oil and furniture polish, and in combination with other chemicals as a vehicle or solvent, but are also known to possess highly dangerous properties if ingested or aspirated. The toxicity of petroleum distillates generally is indirectly proportional to the material's viscosity, with products having higher viscosity being considered to be less toxic. Products with viscosity in the range of 30 to 35 SUS (Saybolt Universal Seconds) or lower present an extreme aspiration risk, and include such agents as mineral oil, found in furniture polishes, mineral spirits, naphthalene, and vegetable oils such as rape seed oil and nut oil. Even small amounts of a low-viscosity material, once aspirated, can involve a significant portion of the lung, and produce a chemical pneumonitis. Oral ingestion of petroleum distillates is often associated with symptoms of mucous membrane irritation, vomiting and central nervous system depression.

Accordingly, restrictions have been placed upon such products as petroleum distillate based furniture polishes, such that non-emulsion liquid furniture polish products containing greater than 10 percent petroleum distillates essentially cannot be legally marketed in a trigger sprayer, at the present time. Since consumers are known to prefer to apply such liquids as cleaning compositions by means of trigger sprayers or pump bottles, it may be anticipated that consumers would welcome such petroleum distillate containing household products as furniture polish, cleaning oils, and the like, in a spray bottle. However, to date, no such products are available which meet the requirements of the Consumer Product Safety Commission (hereinafter CPSC) that effectively require that a petroleum distillate composition for spray application exhibit a viscosity of greater than 100 SUS at 100° F. [16 CFR 1700.14 (a) (2)]. Further, the CPSC has recently approved regulations that require child-resistant packaging for most oil-based household products. The newly approved rules of the CPSC would apply to thin, watery products containing at least 10 percent hydrocarbon by weight, which flow freely and can be inhaled or aspirated when swallowed. Since thicker, more viscous liquids are less likely to be inhaled, these requirements will apparently not apply to such liquids.

Thrasher, U.S. Pat. No. 5,342,436, teaches a liquid phase composition for topical application to wood or other surfaces as a sealant and protective finish. The composition comprises paraffin wax dispersed in mineral oil and an extender, and various optional additives. Thickeners and a solubilizing agent such as a volatile low viscosity silicone may be added to produce a creamy form. The composition may be applied to wood as a furniture polish requiring little or no buffing. This composition clearly has an elevated viscosity, and is applied to the surface by spreading with a sponge, brush, or the like.

Kasprzak, U.S. Pat. No. 4,218,250, teaches a polish formulation containing a wax and/or an abrasive and at least one member of the group consisting of solvents, surfactants, thickening agents, detergent resistant additives, colorants, odorants, and other conventional polish ingredients, to which is added a cyclodimethyl siloxane fluid, a copolymer, and water. The thickening agents suggested as being commonly used in such polish formulations are soya lecithin and methyl cellulose. The resulting improved polishes comprised from 30 to 95 percent water, and were thick non-flowing paste polish compositions applicable by rubbing in place with a dry cloth, or as an aerosol polish composition. Thus, the thickening agents of the patent are effective in thickening the water component of the formulation.

Wantling, in U.S. Pat. No. 6,066,201, teaches a thixotropic wax emulsion composition comprising a wax, an emulsifier, and a naphthenic oil. Such compositions comprise fluids which flow upon shearing as through a pump, returning to the original state or thickening upon cessation of shearing. Naphthenic oils having viscosities between 40 and 5000 SUS at 100° F., are preferred. The compositions of the patent comprise modified slack wax emulsions, although any suitable hydrocarbon waxes may be used. Typical waxes include more than 50 percent slack wax and more than 40 percent water, the remainder being naphthenic oil, wetting agent, soap system, and other control additives. While the composition is thixotropic, there is no teaching that the material could be applied by means of a trigger spray bottle, and in fact, the patent indicates usages inconsistent with such an application method. Again, the thickening agents of the patent are effective in an oil in water emulsion environment, in a wax emulsion.

To date, no petroleum distillate based household products are available which may be dispensed by trigger spray or pump spray means, which are non-emulsion formulations absent water, and which exceed the minimum viscosity requirements of the CPSC. It is therefore considered desirable to provide a means for thickening petroleum distillates in such a manner that they may be safely dispensed by means of a trigger spray bottle.

### BRIEF SUMMARY OF THE INVENTION

To achieve the foregoing and other goals, the present invention provides a liquid phase petroleum distillate product for spray application to a surface, wherein the viscosity of the petroleum distillate liquid composition at rest exceeds 100 SUS at a temperature of 100° F. The composition utilizes a thixotropic, shear-thinning, thickener to provide a liquid of elevated viscosity when at rest. By shear-thinning, it is meant that the composition thins down to a lowered viscosity under shear, and may thus be passed through a nozzle to form a mist when sprayed. Were the composition not subject to shear-thinning, the thickened petroleum distillate would not be able to be passed through a nozzle to form a mist or spray, as in a trigger spray applicator. The liquid then thickens, after spraying and upon removal of shear, to a higher viscosity before coming in contact with the surface toward which it has been sprayed. By providing such a modified petroleum distillate product, the present invention overcomes several problems inherent in conventional petroleum distillate based products such as those containing mineral oil or mineral spirits, often used in such products as furniture polish. Conventional petroleum distillate products of this nature tend to run down vertical surfaces, due to low viscosity, making wiping more difficult. More importantly, such lower viscosity petroleum distillate products are harmful if swallowed. The modified products of the present invention, however, run more slowly down vertical surfaces, and thicken before contacting the back of a person's throat if sprayed in that direction. Moreover, whereas most thin mineral oils are difficult to dispense as a spray from a non-pressurized container, because they can form a stream rather than a mist when dispensed, the thickened mineral oils of the present invention form a mist or spray upon dispensing though a trigger spray or pump dispenser mechanism. As a result of the application of shear forces to the thixotropic thickened composition, the composition thins to a more suitable viscosity, allowing passage through the nozzle of a trigger sprayer or pump dispenser. After passage through the spray nozzle, the composition then thickens again to a more viscous liquid. The method of the present invention, comprising the addition of an effective amount of a shear-thinning thixotropic thickener, to a low viscosity petroleum distillate, is applicable to petroleum distillates suitable for use in such household products as furniture oils; furniture polishes and cleaners; baby oils; sunscreens; nail enamel dryers; hair oils; bath, body and massage oils; makeup removers; some automotive chemicals, such as gasoline additives, fuel injection cleaners, and carburetor cleaners; cleaning solvents; water repellents containing mineral spirits; general household oil; gun-cleaning solvents containing kerosene; and the like.

### DETAILED DESCRIPTION OF THE INVENTION

The present invention comprises the addition of a thixotropic thickening agent to a petroleum distillate composition having a viscosity typically on the order of about 50 SUS at 100° F. Exemplary petroleum distillate products encompass oils commonly employed in household products, as set forth above, and particularly those mineral oils having a viscosity of less than 130 SUS at 100° F., and more preferably those mineral oils having a viscosity of less than 100 SUS at 100° F.

The thickeners found to be of value in this respect comprise thickeners which provide shear-thinning attributes to a petroleum distillate, wherein there is essentially no water present. Thus the present invention is not intended for use with oil-in-water, or water-in-oil emulsions employing petroleum distillates, all though it is theorized that such thickeners may be effective with water-in-oil emulsions wherein the water phase is entirely encapsulated in the oil phase.

A very limited number of such thickeners have been found to be of value in the present invention. One such thixotropic thickener is Versagel™ M 1600, a mineral oil/Kraton® block copolymer composition available from Penreco. Kraton® is a commercially available thermoplastic rubber type polymer, sold by Shell Chemical Company. This oil/copolymer material, as described in U.S. Pat. No. 5,879,694, (incorporated herein by reference) has been used in the past for the manufacture of gel candles, but has now been found to act as a thixotropic thickener when mixed with petroleum distillates in the absence of water. Such block copolymer blends with hydrocarbon oil, such as paraffinic oil, naphthenic oil, natural mineral oil, and particularly white oil, constitute a preferred shear-thinning thixotropic thickener for use with non-aqueous petroleum distillate products.

Another suitable thickener has been found to be N-Lauroyl glutamic acid di-n-butylamide. In one attempt at thickening a mineral oil, addition of this thickener resulted in a viscosity of 2000 SUS at 100° F., but the oil was capable of being sprayed.

Such thixotropic thickeners may be used quite sparingly, since the addition of very small amounts thereof will cause the viscosity of the petroleum distillate to increase rapidly. For example, the addition of 9.00 percent of Versagel™ M 1600 to mineral oil having a viscosity of 50 SUS rapidly results in a thickened composition having a viscosity of 180 SUS. It may be seen that the addition of excessive thickener may result in a composition which, even with shear-thinning properties, will be too viscous to be sprayed. By careful selection of the appropriate thixotropic thickening agent for any specific petroleum distillate, one may thicken the chosen petroleum distillate in such a manner that it may be applied to a surface by means of a trigger spray mechanism, while still maintaining a high viscosity at rest, so as to be packaged in a conventional container without requiring special packaging called for by the CPSC. For purposes of the present invention, it is suggested that from about 0.1 to about 15 percent by weight of the thickener be present in the thickened petroleum distillate product. Preferably, the thickener should be present in an amount of from about 0.2 to about 12 percent by weight, and more preferably from about 0.2 to about 9 percent.

### EXAMPLE 1

To a mixture comprising mineral oil having a viscosity of 50 SUS, and a fragrance, was added 1.0 percent by weight of a thixotropic thickener comprising N-Lauroyl glutamic acid di-n-butylamide. The resulting mixture exhibited a viscosity of 1900 cPs at room temperature. This mixture was then placed in a trigger spray bottle, and was dispensed through the nozzle by activation of the trigger. The composition discharged as a fine mist. The initial blend of mineral oil and fragrance was placed in a similar container having a trigger spray dispenser, absent the thixotropic thickener. When subjected to the trigger action of the dispenser, the liquid discharged as a liquid stream rather than as a spray or mist.

### EXAMPLE 2

A furniture polish composition was prepared in accordance with the following formulation, as set forth in Table A, below. The viscosity of the composition was measured, and found to be 110 SUS at 110° F. This furniture polish composition was placed in a spray bottle with trigger spray mechanism and nozzle, and sprayed upon a table surface. The polish was deposited as a fine mist, and was easily spread to form a uniform coating which dried to a high gloss finish. A small amount of the polish was sprayed upon a surface, collected, evaluated, and found to immediately return to the viscosity it had prior to spraying. Conversely, it was found that a test oil having a viscosity of 60 SUS at 100° F., absent the thixotropic thickener of the Example, was not capable of being dispensed as a spray through the same trigger spray mechanism and nozzle.

TABLE A

| Percentage | Component | Function |
|---|---|---|
| 80.48 | Mineral Oil - 50 SUS | Solvent/Cleaner |
| 8.50 | Versagel ™ M 1600 | Thixotropic thickener |
| 4.00 | d-Limonene | Cleaner/Solvent |
| 5.00 | Silicone Oil - 20 centistokes | Polish |
| | viscosity | |
| 1.00 | Orange Blast RN-2259 | Perfume |
| 0.0020 | Sandoplast Yellow F3G | Azo dye |
| 0.0002 | Cl Solvent Red 27 | Dye |
| 0.0200 | Bitrex ™ Solution (25% in ethanol) | Bittering Agent |
| 1.00 | Mazol ™ PG031K Monooleate | Surfactant/solubilizer |

### Industrial Applicability

The present invention provides for the modification of the viscosity of petroleum distillate based household products, such as furniture oil or baby oil, so as to permit application by means of a spray mechanism. By addition of a suitable thixotropic thickening agent to a petroleum distillate based product, one is able to provide a liquid of elevated viscosity which is capable of being sprayed upon a surface by means of conventional spray apparatus and mechanisms

We claim:

1. A method for increasing the viscosity of a petroleum distillate, said method comprising adding to said petroleum distillate a shear-thinning thixotropic thickener.

2. The method of claim 1, wherein the thixotropic thickener is selected from the group consisting of a mixture comprising from about 2 percent to about 30 percent of a triblock copolymer with from about 70 percent to about 98 percent of a hydrocarbon oil, and N-Lauroyl glutamic acid di-n-butylamide.

3. The method of claim 2, wherein the thixotropic thickener comprises a triblock copolymer, and said thickener comprises from about 0.1 to about 15 percent by weight of the composition.

4. The method of claim 3, wherein the thixotropic thickener further comprises a diblock copolymer.

5. The method of claim 3, wherein the petroleum distillate initially has a viscosity of less than about 130 SUS at 100° F.

6. The method of claim 2, wherein the petroleum distillate is selected from the group consisting of mineral oils, mineral spirits, naphthalene, and vegetable oils.

7. A method for increasing the viscosity of a mineral oil, said method comprising adding to said mineral oil an effective amount of a shear-thinning thixotropic thickener.

8. The method of claim 7, wherein the thixotropic thickener is selected from the group consisting of a mixture comprising from about 2 percent to about 30 percent of triblock copolymer with from about 70 percent to about 98 percent of hydrocarbon oil, and N-Lauroyl glutamic acid di-n-butylamide.

9. The method of claim 8, wherein the thixotropic thickener comprises a triblock copolymer.

10. The method of claim 9, wherein the thixotropic thickener further comprises a diblock copolymer.

11. The method of claim 8, wherein the petroleum distillate has a viscosity of less than about 130 SUS at 100° F.

12. The method of claim 8, wherein the petroleum distillate is selected from the group consisting of mineral oils, mineral spirits, naphthalene, and vegetable oils.

13. A non-aqueous petroleum distillate product suitable for dispensing through a spray mechanism, said product comprising a petroleum distillate and a shear-thinning thixotropic thickener.

14. A product as set forth in claim 13, wherein said thixotropic thickener is selected from the group consisting of a mixture comprising from about 2 percent to about 30 percent of a triblock copolymer with from about 70 percent to about 98 percent of a hydrocarbon oil, and N-Lauroyl glutamic acid di-n-butylamide.

15. A product as set forth in claim 14, wherein said thixotropic thickener is present in an amount of from about 0.2 to about 12 percent by weight of the composition.

16. A product as set forth in claim 15, wherein said petroleum distillate is selected from the group consisting of mineral oils, mineral spirits, naphthalene, and vegetable oils.

17. A product as set forth in claim 16, wherein said petroleum distillate is a mineral oil.

18. A product as set forth in claim 16, selected from the group consisting of furniture oils; furniture polishes and cleaners; baby oils; sunscreens; nail enamel dryers; hair oils; bath, body and massage oils; makeup removers; gasoline additives; fuel injection cleaners; carburetor cleaners; cleaning solvents; water repellents; and general household oil.

19. A product as set forth in claim 18, comprising a furniture oil wherein said petroleum distillate is a mineral oil, and said thixotropic thickener comprises from about 2 percent to about 30 percent of a triblock copolymer and from about 70 percent to about 98 percent of a hydrocarbon oil.

20. A product as set forth in claim 19, wherein said thickener comprises from about 0.2 to about 9 percent by weight of the product.
